(19) United States
(12) Patent Application Publication
(10) Pub. No.: US 2010/0331603 A1
(43) Pub. Date: Dec. 30, 2010

(54) METHOD AND DEVICE FOR THE PHYSICAL TREATMENT OF PARETIC PATIENTS

(76) Inventor: Johann Szecsi, Petershausen (DE)

Correspondence Address: K&L Gates LLP, P.O. Box 1135, CHICAGO, IL, 60690, US

(21) Appl. No.: 12/726,689

(22) Filed: Mar. 18, 2010

Related U.S. Application Data
(60) Provisional application No. 61/161,278, filed on Mar. 18, 2009.

(51) Int. Cl. A61N 2/02; A61N 2/00; A63B 22/06

(52) U.S. Cl. 600/13; 600/9; 482/57

(57) ABSTRACT

The present invention relates to a training device for the physical therapy of paretic patients, comprising at least one magnetic stimulator for applying functional magnetic stimulation to paralyzed muscles of said patient in order to induce a periodical movement; at least one guiding element for restricting the degrees of freedom of the movement induced; and at least one resistance element for providing a resistance against the movement induced, wherein the device is configured such that the torque of the movement induced is at least 1.25 Nm. The invention also concerns a therapy method for a paretic patient, comprising providing such a training device, applying magnetic stimulation, impeding the movement via the at least one resistance element; and determining the torque of the movement induced.

### PRIORITY

This application claims the benefit of U.S. Patent Application Ser. No. 61/161,278 filed Mar. 18, 2009, the disclosure of which is hereby incorporated by reference in its entirety.

### TECHNICAL FIELD OF THE INVENTION

The present invention relates to a training device for the physical therapy of paretic patients, comprising at least one magnetic stimulator for applying functional magnetic stimulation to paretic (in particular, to incompletely paralyzed) muscles of said patient in order to induce a periodical movement; at least one guiding element for restricting the degrees of freedom of the movement induced; and at least one resistance element for providing a resistance against the movement induced, wherein the device is configured such that the torque of the movement induced is at least 1.25 Nm. The invention also concerns a therapy method for a paretic patient, comprising providing such a training device, applying magnetic stimulation, impeding the movement via the at least one resistance element; and determining the torque of the movement induced.

### BACKGROUND OF THE INVENTION

Functional electrical stimulation (FES) is a promising rehabilitation technique for artificially activating muscles that are not under voluntary control following a spinal cord injury (SCI) or a cerebrovascular insult (Gorman, P. H. et al. (2006), in: Neural Repair and Rehabilitation. (Selzer, M. E. et al., Eds.), Cambridge University Press, p. 119-135). Possible applications of FES are to propel or support mobility (gait or cycling) and to make possible conditioning exercises. The advantage of cycling is that it can be maintained for reasonably long periods and the risk of fall is low.

FES-propelled cycling in persons with complete spinal cord injury (SCI) is known to train the cardiovascular system (Glaser, R. M. (1994) Int. J. Spots Med. 15, 142-148), to strengthen the muscles (Mohr, T. et al. (1997) Calcif Tissue Int. 61, 22-25), as well as to improve cycling mobility (Perkins, T. A. et al. (2002) IEEE Trans. Neural. Syst. Rehabil. Eng. 10, 158-164; Hunt, K. J. et al. (2004) IEEE Trans. Neural. Syst. Rehabil. Eng. 12, 89-101). Exemplary training devices employing FES have been disclosed inter alia in U.S. Pat. No.4,499,900 as well as in U.S. patent applications 2003/0109814 A1 and 2005/0015118 A1, respectively.

Mostly, FES cycling focuses on patients with complete SCI, although the stroke population is approximately 10-fold that of the SCI population (Kirsch, R. F. and Kilgore. K. L. (2004). in: Neuroprosthetics. (Horch, K. W., and Dhillon, G. S., Eds.), First ed. New Jersey: World Scientific. p. 981-1004).

It is thought that electrical stimulation can also be used in the latter case for training purposes as well as for achieving ultimate functional improvement. However, FES appears clinically impractical in the stroke population, because it induces pain (Liberson, W. T. et al. (1961) Arch. Phys. Merl. Rehabil. 42, 101-105; Takebe, K. et al. (1975) Arch. Phys. Med. Rehabil. 56, 237-239) due to unavoidable stimulation of the skin receptors, including A-delta myelinated heat nociceptors and C-fiber nociceptors (Adriaensen, H. et al. (1983) J. Neurophysiol. 49, 111-122; Chae, J. et al. (1998) Am. J. Phys. Med. Rehabil. 77, 516-522).

For this reason, 8 of 46 subjects in a study on the efficacy of FES in acute stroke patients could not tolerate FES treatment (Chae, J. et al. (1998) Stroke 29, 975-979). In the study of Yan and colleagues (Yan, T. et al. (2005) Stroke 36, 80-85) thigh stimulation intensities of 20-30 mA were used to achieve weight-supported knee joint movement. In two studies on leg stimulation-supported gait in the same group (Tong, R. K. et al. (2006a) Arch. Phys. Med. Rehabil. 87, 1298-1304; Tong, R. K. et al. (2006b) Phys. Ther. 86. 1282-1294) the stimulation intensity was set to 50-85 mA in an effort to achieve limb movement at the subject's comfort threshold. However, only small or sub-maximal isometric torques could be generated at those intensities as seen in the torque recruitment curve of the quadriceps. It has been shown that increases in quadriceps femoris strength in a normal population who trained with FES correlated with training contraction intensity and duration (Selkowitz, D. M. (1985) Phys. Ther. 65, 186-196). It was concluded that the relative increase in isometric strength resulting from training with FES might be determined by the ability of the subjects to tolerate longer and more forceful contractions. Another study also demonstrated that cycling power and smoothness in acute stroke patients are limited by the individual's ability to tolerate stimulation current (Szecsi, J. et al. (2008) Clin. Biomech. 23, 1086-1094).

In contrast, by using time-varying electromagnetic fields to induce eddy currents in the adjacent volume without passing the skin, repetitive functional magnetic stimulation (FMS) activates the nerve innervating the muscle without stimulating the skin nociceptors (Barker, A. T. et al. (1987) Neurosurgery 20, 100-109; Barker, A. T. (1991) J. Clin. Neurophysiol. 8, 26-37). Moreover, magnetic stimulation does not produce radial current, which activates pain nerves in the skin best (Cohen, D. and Duffin, B. N. (1991) J. Clin. Neurophysiol. 8, 102-111).

However, the application of FMS is hampered by the fact that, as compared to electrical stimulators, magnetic stimulators are bulkier and they cannot provide focal stimulation (Cohen, D. and Duffin, B. N. (1991), supra). This is a significant drawback because human movement, particularly cycling, is equally dependent on isometric force and power output (Newham, D. J. and Donaldson, N. N. (2007) Acta Neurochir. Suppl. 97, 395-402). The available reports on magnetic stimulation to generate muscle force in legs of normal persons are very rare (Han, T. R. et al. (2006) Am. J. Phys. Med. Rehabil. 85, 593-539; Kremenic, I. J. et al. (2004) Muscle Nerve 30, 379-381), and there are no reports at all on the generation of power in persons with only partially preserved or lost sensibility.

Thus, there still remains a need for training devices and corresponding treatment regimens for the physical therapy of paretic patients, and in particular for hemi-paretic patients having an at least partially preserved sensibility in the paralyzed part of the body, that overcome the above-limitations.

More specifically, there is a need for devices and methods allowing for a more efficient training intensity as compared to known treatment systems without causing inconvenience for the patients.

### SUMMARY OF THE INVENTION

In a first aspect, the present invention relates to a method for the physical therapy of a paretic patient, the method comprising:
(a) providing a training device, comprising
   (i) at least one magnetic stimulator for applying functional magnetic stimulation to at least one paralyzed muscle or muscle group of said patient in order to induce a periodical movement;
   (ii) at least one guiding element for restricting the degrees of freedom of the movement induced by applying functional magnetic stimulation; and
   (iii) at least one resistance element for providing a resistance against the movement induced by applying functional magnetic stimulation;
(b) applying magnetic stimulation to at least on paralyzed muscle or muscle group of said patient in order to induce a movement;
(c) impeding the movement of said muscle or muscle group via the at least one resistance means; and
(d) determining the torque of the movement induced.

Preferably, the periodical movement is a cyclic movement.

In another preferred embodiment, the method further comprises:
(e) adjusting the functional magnetic stimulation in such a manner that the torque of the movement induced is at least 1.25 Nm.

In a further preferred embodiment, the patient to be treated is a patient having an at least partially preserved sensibility in the paralyzed part of the body. In one specific embodiment, the patient to be treated is a patient suffering from a condition selected from the group consisting of stroke, multiple sclerosis, cerebral paresis, and traumatic brain injury, and complete (SCI-A) or incomplete (SCI-B, C, D) spinal cord injury.

In a further specific embodiment of the method, the at least one guiding means is selected from the group consisting of a stationary cycle, an ergometer, a cross-trainer, a rowing machine, a robot, and an exoskeleton.

In another preferred embodiment of the method, the at least one magnetic stimulator comprises any one or more of the group consisting of (i) one or more coil(s) selected from the group consisting of a ring coil, an elliptic coil, a saddle coil, a sleeve coil, a manchette coil or a semicylindrical coil, (ii) an acoustic attenuation or damping means: and (iii) a cooling system, the cooling system being configured to allow the continuous operation of the at least one magnetic stimulator for at least 5 minutes at a frequency of at least 20 Hz.

In a specific embodiment, the one or more coil(s) of the at least one magnetic stimulator is/are arranged in a latero-ventral position relative to the at least one paralyzed muscle or muscle group to be stimulated.

In another specific embodiment of the method, the one or more coil(s) of the at least one magnetic stimulator is/are configured to apply magnetic stimulation to a body surface area of at least 250 cm². The body surface preferably covers the quadriceps muscle group of the patient to be treated.

In one embodiment of the method, the functional magnetic stimulation is continuously applied to the at least one paralyzed muscle or muscle group of the patient for at least 5 minutes during a treatment regimen.

In an alternative embodiment of the method, the functional magnetic stimulation is applied in at least three bouts within one day to the at least one paralyzed muscle or muscle group of the patient during a treatment regimen, each of the at least three bouts comprising a continuous application for at least two minutes.

In a second aspect, the present invention relates to a training device for the physical therapy of a paretic patient, comprising:
(a) at least one magnetic stimulator for applying functional magnetic stimulation to at least one paralyzed muscle or muscle group of said patient in order to induce a periodical movement;
(b) at least one guiding element for restricting the degrees of freedom of the movement induced by applying functional magnetic stimulation; and
(c) at least one resistance element for providing a resistance against the movement induced by applying functional magnetic stimulation;
   wherein the device is configured in such a manner that the torque of the movement induced is at least 1.25 Nm.

In a further specific embodiment of the device, the at least one guiding means is selected from the group consisting of a stationary cycle, an ergometer, a cross-trainer, a rowing machine, a robot, and an exoskeleton.

In a preferred embodiment of the device, the at least one magnetic stimulator comprises any one or more of the group consisting of (i) one or more coil(s) selected from the group consisting of a ring coil, an elliptic coil, a saddle coil, a sleeve coil, a manchette coil or a semicylindrical coil; (ii) an acoustic attenuation or damping means; and (iii) a cooling system, the cooling system being configured to allow the continuous operation of the at least one magnetic stimulator for at least 5 minutes at a frequency of at least 20 Hz.

In a specific embodiment, the one or more coil(s) of the at least one magnetic stimulator is/are arranged in a latero-ventral position relative to the at least one paralyzed muscle or muscle group to be stimulated. Preferably, the one or more coils are arranged at a distance of at least 5 mm from the body surface area where the stimulation has to be applied

In another specific embodiment of the device, the one or more coil(s) of the at least one magnetic stimulator is/are configured to apply magnetic stimulation to a body surface area of at least 250 cm². The body surface preferably covers the quadriceps muscle group of the patient to be treated.

Other embodiments of the present invention will become apparent from the description hereinafter.

### BRIEF DESCRIPTION OF THE DRAWINGS

FIG. 1 depicts an isometric and cycling measurement setup according to the invention. Both FMS and FES stimulation are possible. A subject with SCI-A performing FMS-propelled cycling using two magnetic stimulators can be seen: (1) torque transducer, (2) angular encoder, (3) right side repetitive magnetic stimulator, (4) left side coil (4, 5) clamps made of foam and Velcro straps. Inset: definition of the crank angle

FIG. 2 depicts isometric measurements performed on a representative subject belonging to the stroke group. Starting from the motor threshold, stepwise-increasing FES and FMS burst amplitudes were applied, until maximum tolerable intensity was reached, in the first and the last part of the protocol, respectively. Combined stimulation (the FMS burst sequence was superimposed on the FES) was applied in the middle part of the protocol.

FIG. 3 depicts isometric measurements performed on a representative subject belonging to the SCI group. In the first part of the test, starting from the motor threshold, stepwise-increasing FMS burst amplitudes were used until maximal intensity was reached. In the second part of the test, combined stimulation was applied (the FMS burst sequence was superimposed twice on the FES).

FIG. 4 depicts cadence (upper graph) and crank torque (lower graph) profiles measured in a subject of the stroke group with right hemiparesis during volitional (gray) and FMS-supported volitional (black) cycling conditions taken over 15 s periods. Measurement points and 10th order polynomial fitting curve are represented. (QUAD): stimulation interval of the right quadriceps.

FIG. 5 depicts isometric torque, power, smoothness, and symmetry of n=29 chronic post-stroke hemiplegic patients measured under volitional, FMS, and FES stimulation conditions. NOTE: Bars and segments plotted represent group means±SD. *FMS compared to FES with significance of p<0.05; ## Stimulation compared to volitional with significance of p<0.001

FIG. 6 depicts the isometric torque and power of n=11 chronic SCI-A patients measured under FMS and FES stimulation conditions. NOTE: Bars and segments plotted represent group means±SD. Asterisks represent significant comparisons of FES and FMS: *p−0.003; **p<10⁻⁴, respectively.

FIG. 7 depicts the distribution of the torque generated by FES (M & M, left panel) or FMS (Magstim, right panel) depending on the site(s) of stimulation. The data represent normalized mean values based on 26 patients. Electrical or magnetic stimulation (of the same intensity) was applied to the thigh of the patients at nine different locations according to the picture insert. With FES, placing one electrode strictly ventral at the groin line and the other one proximal to the kneecap results in the highest efficacy. In contrast, with FMS, arranging the magnetic stimulator in a latero-ventral position relative to the surface area, where the stimulation is applied, shows optimal results.

FIG. 8 depicts the dependency of the efficacy generated by FES or FMS on the size of the body surface area, to which the stimulation is applied, both with regard to the pain perception/intensity of the patients treated (AUC, area under the curve; left panel) and the maximal torque determined (right panel). The data represent normalized mean values based on 26 patients. The following set-ups were used: FES-K (positioning of the electrodes close to each other, at a distance of about 13 cm, that is, according to the typical diameter of a round magnetic coil); FES-L (standard arrangement of the electrodes as described in FIG. 6); FMS-L (standard round magnetic coil, diameter 13 cm); and FMS-S (magnetic saddle coil with substantially elliptic guidance, the coil having a length of about 30 cm and a width of about 20 cm; or manchette (collar, sleeve) coil, e.g. by Magstim Company Ltd., Spring Gardens, Whitland Carmarthenshire, Wales, UK).

FIG. 9 depicts the same experiment as FIG. 8. The picture inserts illustrate the respective types of stimulator means and the experimental set-ups employed. As apparent from the graphs, the use of FMS-S results not only in a reduced pain perception but also in the generation of an increased maximal torque, as compared to the remaining set-ups.

FIGS. 10-12 schematically illustrate a further embodiment of a training device according to the invention. FIG. 11 represents an overall illustration of the training device. The patient to be treated (suffering from paralysis in his legs) is positioned in the training device in an upright (standing) position fixed with a chest strap and by means of two grip bars for his hands. FMS is applied to his thighs via magnetic stimulators having manchette coils (collar coils, sleeve coils) that cover the entire thighs (FIG. 12). The magnetic stimulators are controlled via stimulation channels that are connected with a computer unit for controlling and/or coordinating the movement induced. The exiting current of a stimulator is redirected to the manchette coils by means of a power-switch controlled by a computer unit (FIG. 10).

FIG. 13 depicts another embodiment of a training device according to the present invention. It comprises a large surface (at least 400 cm²) saddle-shaped coil with outer dimensions of 31 cm×20 cm, an inner cylindrical surface, and an aperture angle of 140°.

### DETAILED DESCRIPTION OF THE INVENTION

The present invention is based on the unexpected finding that the use of large-area magnetic stimulators as integrative components of specifically configured training devices for the physical therapy of paretic patients results in significantly improved training results as compared to both functional electrical stimulation and functional magnetic stimulation employing conventional magnetic stimulators. More specifically, the therapy can be performed with an increased intensity and without causing inconveniences for the patients to be treated such as significant pain perception or skin irritations due to the direct application of electrodes to the body parts to be stimulated.

The present invention illustratively described in the following may suitably be practiced in the absence of any element or elements, limitation or limitations, not specifically disclosed herein. The present invention will be described with respect to particular embodiments and with reference to certain drawings but the invention is not limited thereto but only by the claims. The drawings described are only schematic and are to be considered non-limiting.

Where the term “comprising” is used in the present description and claims, it does not exclude other elements or steps. For the purposes of the present invention, the term “consisting or” is considered to be a preferred embodiment of the term “comprising of”. If hereinafter a group is defined to comprise at least a certain number of embodiments, this is also to be understood to disclose a group, which preferably consists only of these embodiments.

Where an indefinite or definite article is used when referring to a singular noun e.g. “a” or “an”, “the”, this includes a plural of that noun unless specifically stated otherwise.

The term “about” in the context of the present invention denotes an interval of accuracy that the person skilled in the art will understand to still ensure the technical effect of the feature in question. The term typically indicates deviation from the indicated numerical value off ±10%, and preferably ±5%.

Furthermore, the terms first, second, third, (a), (b), (c), and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Further definitions of term will be given in the following in the context of which the terms are used. These terms or definitions are provided solely to aid in the understanding of the invention. These definitions should not be construed to have a scope less than understood by a person of ordinary skill in the art.

In a first aspect, the present invention relates to a method for the physical therapy of a paretic patient, the method comprising:
(a) providing a training device, comprising
   (i) at least one magnetic stimulator for applying functional magnetic stimulation to at least one paralyzed muscle or muscle group of said patient in order to induce a periodical movement;
   (ii) at least one guiding element for restricting the degrees of freedom of the movement induced by applying functional magnetic stimulation; and
   (iii) at least one resistance element for providing a resistance against the movement induced by applying functional magnetic stimulation;
(b) applying magnetic stimulation to at least on paralyzed muscle or muscle group of said patient in order to induce a movement;
(c) impeding the movement of said muscle or muscle group via the at least one resistance element; and
(d) determining the torque of the movement induced.

The term “paretic patient” (herein also referred to as “paralytic patient”), as used herein, relates to subjects suffering from an at least partial paralysis of at least one muscle or muscle group of their body. The term “paralysis”, as used herein, denotes the partial or complete loss of muscle function for one or more muscle groups. Paralysis can cause loss of feeling or loss of mobility in the affected area such as one or both legs or arms.

Etiologically, paralysis may be caused by various types of medical conditions. Most often, it is caused by damage to the nervous system or brain, especially the spinal cord. Major causes are stroke trauma, poliomyelitis, amyotrophic lateral sclerosis (ALS), botulism, spina bifida, multiple sclerosis, and Guillain-Barré syndrome. Temporary paralysis may occur during REM sleep, and dysregulation of this system can lead to episodes of waking paralysis. Drugs that interfere with nerve function, such as curare, may also cause paralysis. Many causes of this are varied, and could also be unknown. Paralysis may be localized, or generalized, or it may follow a certain pattern. Most paralyses caused by nervous system damage are constant in nature; however, there are forms of periodic paralysis as well, including sleep paralysis.

In some embodiments of the present invention, the patient to be treated is a patient suffering from complete (i.e. spinal cord injury type A; SCI-A) or incomplete (i.e. spinal cord injury types B, C, and D; SCI-B, C, D) spinal cord injury. Typically, such patients suffer from a partial or complete loss of muscle function in their legs (i.e. either one or both), that is, these patients are characterized by paraplegia. They also have a partial or complete loss of sensation in their legs (i.e. the paralyzed muscles of their legs).

In preferred embodiments, the patient to be treated is a patient having an at least partially preserved sensibility in the paralyzed part of the body. In other words, such patient still have some remaining mobility in the affected paralyzed part of the body as well as a partial pain perception, and the like. Particularly preferably, the patient to be treated is a patient suffering from a condition selected from the group consisting of stroke, multiple sclerosis, cerebral paresis, and traumatic brain injury. All these medical conditions are well known in the art.

The training devices described herein are configured to enforce a periodical movement, preferably a cyclic movement, of the paralyzed muscles or muscle groups of the patient to be treated. The term “enforcing”, as used herein, is to be understood that it is not sufficient to merely induce a movement (i.e. a contraction of the muscle fibers, followed by a relaxation) by applying functional magnetic stimulation (FMS) to the paralyzed muscles by means of at least one magnetic stimulator but that it is also required to guide the movement, that is, to restrict the degrees of freedom of said movement in such a manner that the movement follows a predetermined course or pattern, preferably a closed pattern, thus resulting in cycling. It is know that cyclic movements (e.g., on a rowing machine or on a bicycle ergometer) result in superior training results.

This goal is accomplished by providing a training device comprising at least one guiding element (i.e. a component or unit of the device as defined herein that is configured for guiding the movement induced by applying functional magnetic stimulation). The type of guiding element selected for a particular therapeutic regimen depends on several factors such as the severity of the paralysis, the part of the body affected (e.g., legs or arms), the overall condition of the patient to be treated, and the like. All this parameters are well known in the art. It is within the professional skills of the physician or medical personnel responsible for the design of the physical therapy to select the most appropriate training device for a particular patient.

In preferred embodiments, the at least one guiding element is selected from the group consisting of a stationary cycle, an ergometer, a cross-trainer, a rowing machine, a robot, and an exoskeleton. Any such guiding elements are well known in the art and commercially available from different suppliers.

Furthermore, in order to improve the therapy outcome the training devices according to the present invention comprise at least one resistance element (i.e. a component or unit of the device as defined herein) for providing a resistance against the movement induced by applying functional magnetic stimulation. One common type of resistance element is a break whose configuration will vary with the type of training device used. The skilled person is well aware of various other types of resistance elements.

The at least one magnetic stimulator may have any configuration as long as it is suitable to apply functional magnetic stimulation to at least one muscle or muscle group. Preferred magnetic stimulators according to the invention are manufactured by Magstim Company Limited, Spring Gardens, Whitland Carmarthenshire, Wales, UK (e.g. the Magstim Rapid stimulator with Booster Setup) or by MAG & More GmbH, Munich, Germany.

In preferred embodiments, the at least one magnetic stimulator comprises one or more coil(s) selected from the group consisting of a ring coil, an elliptic coil, a saddle coil, a sleeve coil, a manchette coil or a semicylindrical coil, with sleeve coils or manchette coils being particularly preferred. In some specific embodiments, the at least one magnetic stimulator comprises at least two coils.

In some embodiments, more than one magnetic stimulator is used, for example two repetitive magnetic stimulators. The maximal magnetic induction applied to the paralyzed muscle or muscle groups is typically between 0.1 T and 10 T, preferably between 0.5 T and 5 T, and particularly preferably between 1 T and 3 T (e.g. 1.2 T, 1.5 T, 1.8 T, 2.0 T, 2.2 T, 2.5 T, and 2.8 T). The duration of the pulses is between 10 μs and 1000 μs (=1 ms), preferably between 50 μs and 500 μs, and particularly preferably between 100 μs and 200 μs. The pulse frequency used is typically between 10 Hz and 100 Hz, even though smaller (e.g. 1 Hz, 3 Hz, 5 Hz) and larger values (e.g. 120 Hz, 150 Hz, 200 Hz, 250 Hz, and so forth) are possible as well. Preferred frequency ranges are between 15 Hz and 30 Hz, 30 Hz and 50 Hz, and 50 Hz to 100 Hz, with the first one being particularly preferred. Most preferred frequencies to be used in the invention are 20 Hz, 25 Hz, and 30 Hz. The stimulation burst durations were chosen according to the maximally tolerable coil heating.

In other preferred embodiments, the at least one magnetic stimulator further comprises an acoustic attenuation or dampening element. Acoustic attenuation refers to the process of making devices less noisy by dampening vibrations to prevent them from reaching the observer. Dampening is achieved by absorbing the vibrational energy or minimizing the source of the vibration, for example by means of providing suitable insulation materials. Numerous acoustic attenuation elements are known in the art (cf. e.g., U.S. Pat. No. 6,386,134) and commercially available from many suppliers.

In further preferred embodiments of the method according to the invention, the at least one magnetic stimulator further comprises a cooling system, the cooling system being configured to allow the continuous operation of the at least one magnetic stimulator for at least 5 minutes (e.g. 8 min, 10, min, 15 min, and so forth) at a frequency of at least 20 Hz. In some embodiments, the cooling system is configured to allow the continuous, operation of the at least one magnetic stimulator for at least 20 minutes (e.g., 25 min, 30 min, 45 min, 60 min, and so forth) at a frequency of at least 20 Hz. However, in other embodiments, the cooling system employed is configured to allow the continuous operation of the at least one magnetic stimulator for a shorter (e.g., 2 min, 5 min or 10 min) time period at a frequency of at least 20 Hz. In still other embodiments, the cooling system employed is configured to allow the continuous operation of the at least one magnetic stimulator for at least 5 minutes (e.g., 8 min, 10 min, 15, min, 25 min, 30 min, 45 min, 60 min, and so forth) at a lower (e.g., 5 Hz or 10 Hz) or a higher frequency (e.g., 25 Hz or 30 Hz). Means for cooling magnetic coils are well established in the art and may be commercially obtained from different vendors.

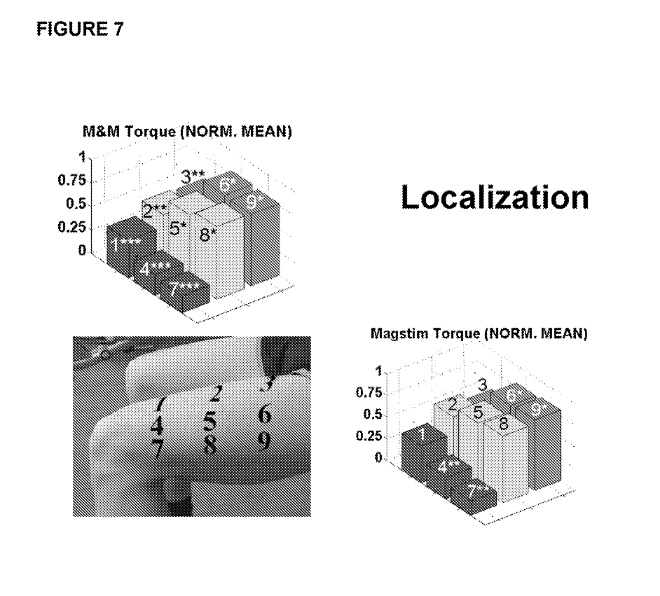

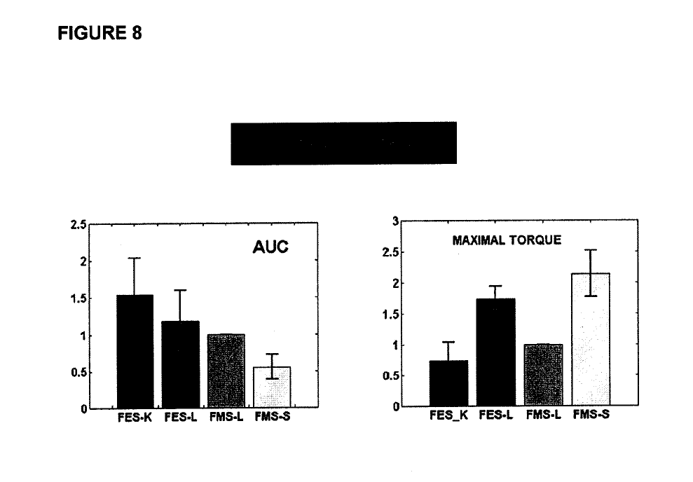

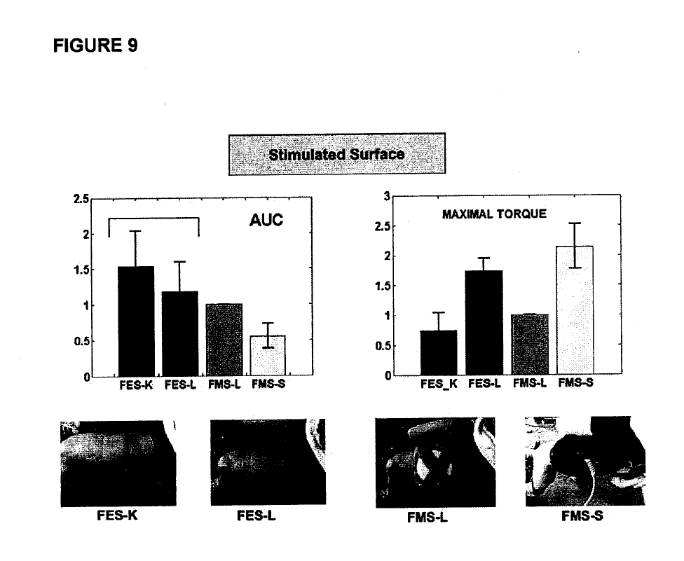

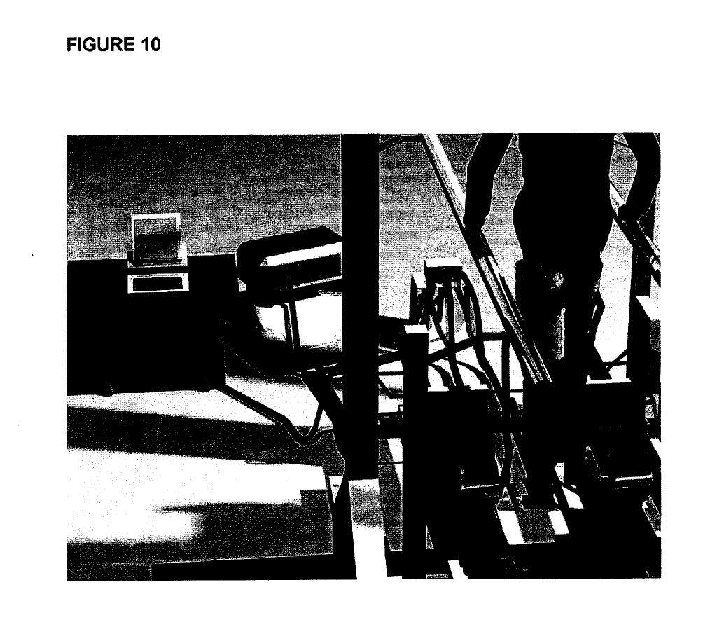

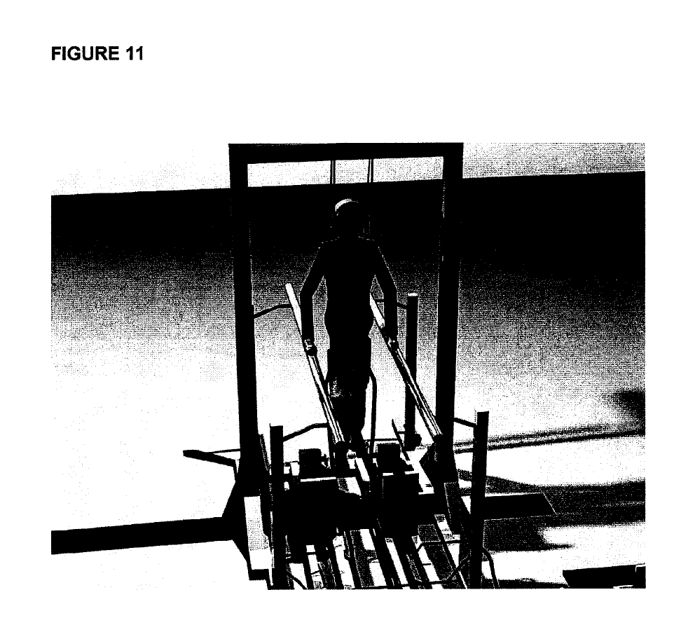

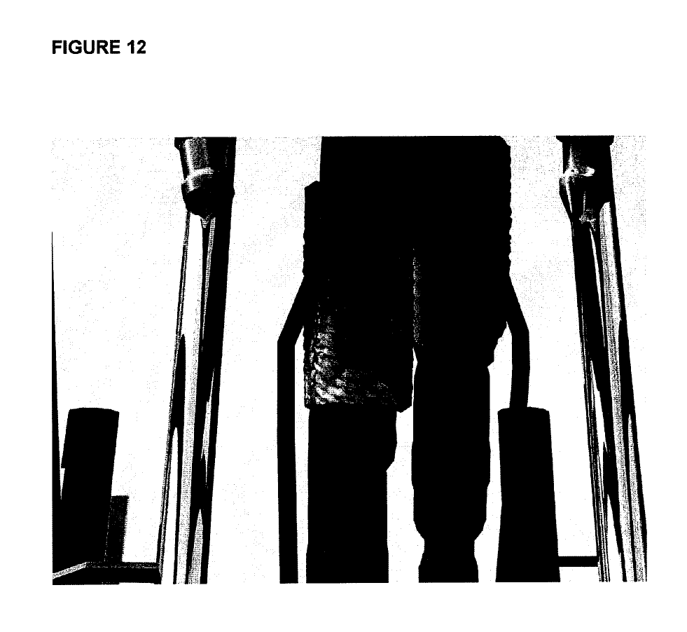

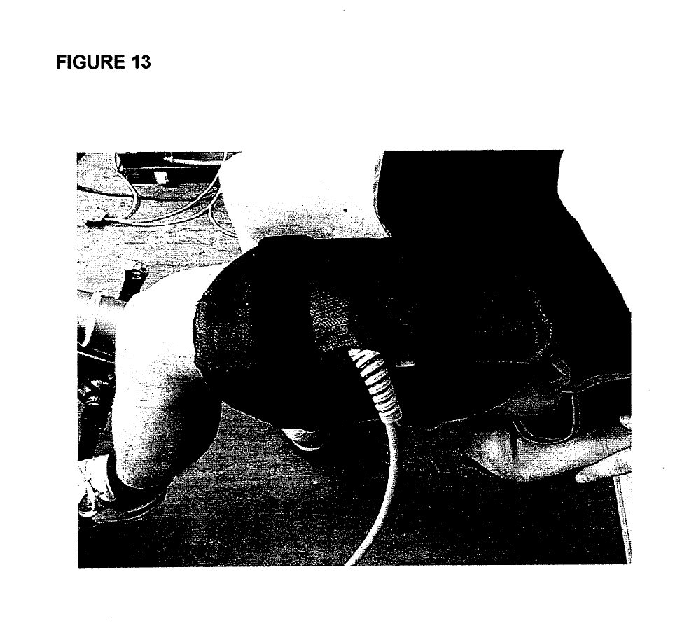

In other preferred embodiments, the one or more coil(s) of the at least one magnetic stimulator is/are arranged in a latero-ventral position relative to the at least one paralyzed muscle or muscle group to be stimulated (cf. FIGS. 7, 9, and 13). In specific embodiments, the coil is placed with its central axis 45° tilted with respect to the frontal plane. Particularly preferably, the one or more coil(s) of the at least one magnetic stimulator is/are configured to apply magnetic stimulation to a (patient's) body surface area of at least 250 cm², that is, the at least one magnetic stimulator covers a respective surface area of at least 250 cm². In other embodiments, the magnetic stimulation is applied to a body surface area of at least 300, 350, 400 cm², 450 cm², 500 cm², 600 cm², 700 cm² or 800 cm². However, larger surface areas are possible as well. In specific embodiments, magnetic stimulation is applied to a body surface area of at least 400 cm². To this end, large surface flexible or semicylindrical coils may be applied.

In specific embodiments, the magnetic stimulation is applied to the thigh(s) of a patient, wherein the body surface to which the stimulation is applied covers the quadriceps muscle group (i.e. the rectus femoris, vastus intermedius, vastus medialis, and vastus lateralis) of the patient to be treated. In other words, the magnetic flow (and thus the induced electric field) produced by the one or more coils of the at least one magnetic stimulator is focused on the quadriceps muscle group of the thigh musculature. In further embodiments, the body surface covers—in addition to the quadriceps muscle group—also the hamstring musculature and/or the gastronemius musculature and/or the tibialis anterior musculature.

Particularly preferably, the one or more coils of the at least one magnetic stimulator are arranged at a distance of at last 5 mm from the body surface area where the stimulation has to be applied. In specific embodiments, the distance is 0.5 mm, 1 mm, 1.5 mm, 2 mm, 2.5 mm, 3 mm, 3.5 mm, 4 mm, and 4.5 mm. However, in other embodiments distances of more than 5 mm are possible as well.

In a further preferred embodiment, the method further comprises: (e) adjusting the functional magnetic stimulation in such a manner that the torque of the movement induced is at least 1.25 Nm. The torque of the movement induced is determined as a measure of the power generated by the functional magnetic stimulation applied. During the treatment regimen, the torque may be adjusted to a given value in order to ensure sufficient training intensity. In preferred embodiments, the functional magnetic stimulation is adjusted in such a manner that the torque of the movement induced is at least 1.5 Nm, 2 Nm, 2.5 Nm, 5 Nm, 7.5 Nm, 10 Nm, or 20 Nm.

In one preferred embodiment, the method of the invention is applied in a physical therapy (i.e. neural rehabilitation), where the functional magnetic stimulation is continuously applied to the at least one paralyzed muscle or muscle group of the patient for at least 5 minutes (e.g. 8 min, 10 min, 15 min, and so forth) during a treatment regimen. In some embodiments, the functional magnetic stimulation is continuously applied to the at least one paralyzed muscle or muscle group of the patient for at least 20 minutes (e.g., 25 min, 30 min, and so forth) during a treatment regimen. Such therapy is particularly suitable for the treatment of patient suffering from complete or incomplete spinal cord injury.

In another preferred embodiment, the method of the invention is applied in a physical therapy where the functional magnetic stimulation is applied in at least three bouts within one day to the at least one paralyzed muscle or muscle group of the patient during a treatment regimen, each of the at least three bouts comprising a continuous application for at least two minutes. Such therapy is particularly suitable for the treatment of patients having an at least partially preserved sensibility in the paralyzed part of the body (e.g., patients suffering from stroke, multiple sclerosis, cerebral paresis, or traumatic brain injury).

In a second aspect, the present invention relates to a training device for the physical therapy of a paretic patient, comprising:
(a) at least one magnetic stimulator for applying functional magnetic stimulation to at least one paralyzed muscle or muscle group of said patient in order to induce a periodical movement;
(b) at least one guiding element for restricting the degrees of freedom of the movement induced by applying functional magnetic stimulation; and
(c) at least one resistance element for providing a resistance against the movement induced by applying functional magnetic stimulation;
   wherein the device is configured in such a manner that the torque of the movement induced is at least 1.25 Nm.

The individual components of the device (e.g., magnetic stimulator, guiding element, resistance element) as well as the mode of operating such a device are as described above in the context of the method according to the invention.

A device as defined herein may be used for the physical therapy of a paretic patient.

Preferably, the patient to be treated is a patient having an at least partially preserved sensibility in the paralyzed part of the body. Particularly preferably, the patient to be treated is a patient suffering from a condition selected from the group consisting of stroke, multiple sclerosis, cerebral paresis, and traumatic brain injury, and complete (SCI-A) or incomplete (SCI-B, C, D) spinal cord injury.

The training device as defined herein may be used for a physical therapy where the functional magnetic stimulation is continuously applied to the at least one paralyzed muscle or muscle group of the patient for at least 5 minutes (e.g., 8 min, 10 min, 15 min, 20 min, 25 min, 30 min, and so forth) during a treatment regimen. In some embodiments, the magnetic stimulation is continuously applied for at least 20 min. Such therapy is particularly suitable for the treatment of patient suffering from complete or incomplete spinal cord injury.

The training device as defined herein may also be used for a physical therapy where the functional magnetic stimulation is applied in at least three bouts within one day to the at least one paralyzed muscle or muscle group of the patient during a treatment regimen, each of the at least three bouts comprising a continuous application for at least two minutes. Such therapy is particularly suitable for the treatment of patients having an at least partially preserved sensibility in the paralyzed part of the body (e.g., patients suffering from stroke, multiple sclerosis, cerebral paresis, or traumatic brain injury).

The invention is further described by the figures and the following examples, which are solely for the purpose of illustrating specific embodiments of this invention, and are not to be construed as limiting the scope of the invention in any way.

### Examples

### Example 1

### A Comparison of Functional Electrical and Magnetic Stimulation for Propelled Cycling of Paretic Patients

1.1. Subjects

(A) Stroke group: Twenty-nine subjects (14 female/15 male; age: 65.1±10.1 years) with chronic post-stroke hemiparesis (16.6±5.5 months post stroke) in a stable condition took part in the study. Mobility ranged from impaired to wheelchair confinement (functional ambulation category FAC 1.86±1.1). Most of these subjects were not able to stand independently and were therefore considered unsuitable candidates for treadmill therapy (Malezic, M. and Hesse, S. (1995) Paraplegia 33, 126-131). Moderately increased muscle tone during knee extension was obvious in all hemiparetic subjects (Modified Ashworth Scale MAS 1.0±0.7).

(B) SCI group: Eleven otherwise healthy subjects (3 female/8 male; age 46.8±12.1 years) with chronic (10.9±8.1 years since injury) SCI grade A and low levels of muscle spasm (MAS range, 0-2) participated in this study. The muscle fiber composition of their paralyzed muscles was stable (Castro, M. J. et al. (1999) J. Appl. Physiol. 86, 350-358).

All subjects were able to comprehend simple instructions. The University of Munich ethics committee had approved the study, and the subjects gave their informed consent prior to their participation.

1.2. Study Design

Each subject underwent three different experimental sessions: (1) isometric measurements using FES and FMS, (2) ergometry using FES, and (3) ergometry using FMS. The session order was randomized, and the three types of experiments were performed first in the SCI group and later in the stroke group. Each session was performed on a different day over a period of 6 weeks (SCI group) and 15 weeks (stroke group).

1.3. Electrical Stimulation

The quadriceps and hamstrings muscle groups were electrically stimulated during ergometric cycling (in the stroke group only on the affected side). For isometric measurements only the left quadriceps group was stimulated in the SCI group and only the affected side in the stroke group. Pairs of auto-adhesive gel electrodes (4.5×9.5 cm² in size; Flextrode, obtained from Krauth & Timmermann Ltd., Hamburg, Germany) were placed on the skin over the proximal and distal fourth of each muscle bulk (similar to Benton, L. A. et al. (1981), supra; Han, T. R. et al. (2006), supra; Noel, G. and Belanger, A. Y. (1987) Physiotherapy Canada 39, 377-383).

A constant-current 8-channel stimulator (Motionstim 8 channel stimulator, Krauth & Timmermann Ltd., Hamburg, Germany) provided the stimulation current (rectangular, biphasic, charged balanced pulses; frequency 20 Hz; maximum pulse amplitude, 127 mA; constant pulse width, 300 μs).

1.4. Magnetic Stimulation

Two repetitive magnetic stimulators were used. The Magstim Rapid stimulator (Magstim Company Ltd., Whitland Carmarthenshire, Wales, UK) provided double cosine pulses (two cosine half-periods, each with 125 μs pulse width) and with 2 Tesla maximal magnetic induction. The P-Stim 160 magnetic stimulator (P-Stim 160 magnetic Stimulator, MAG & More GmbH; München, Germany) generated double cosine pulses (each with 160 μs pulse width) and with 1 Tesla maximal magnetic induction. The frequency was 20 Hz, the same as in electrical stimulation.

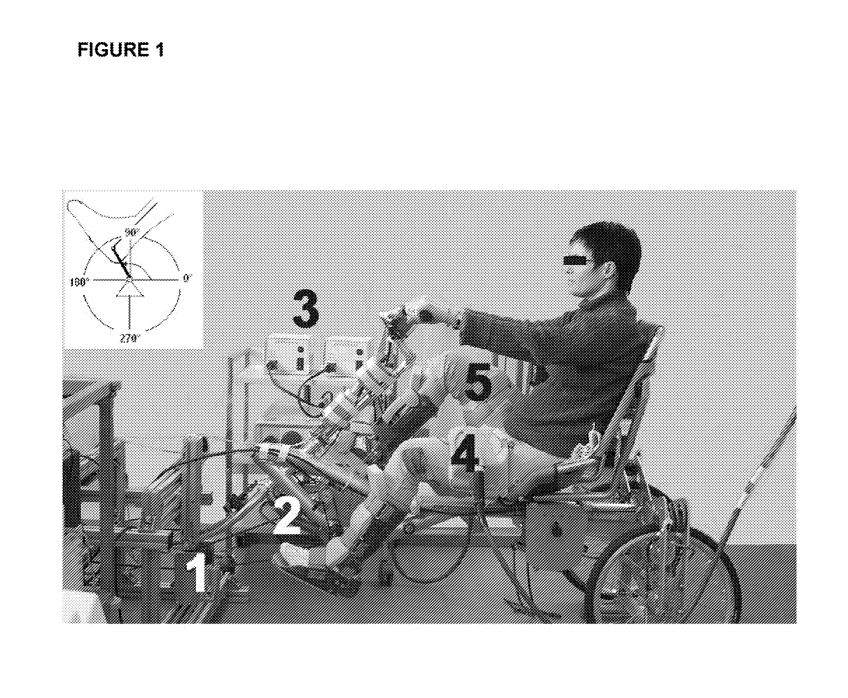

Two round magnetic coils (diameter 90 mm, 23.3 μH inductance; Magstim Company Ltd., Whitland Carmarthenshire, Wales, UK) were placed on the subject's clothes overlying the quadriceps muscle and were tilted 45° to the frontal plane. They were fastened to the proximal half of the muscle bulk by clamps made of foam and Velcro straps (FIG. 1). The stimulation burst durations (see hereinafter) were chosen according to the maximally tolerable coil heating.

During the isometric measurements and ergometric cycling the electrical and the magnetic stimulators were controlled from a personal computer by serial communication. The muscle stimulator was directed to induce muscle contractions on both sides in the SCI group in order to propel cycling and on the affected side in the stroke group, to support volitional pedaling. Muscle contractions were induced at the appropriate crank angles (Perkins, T. A. et al. (2002), supra).

1.5. Isometric Measurements

A stationary tricycle with its front wheel replaced by a torque transducer (T30FN torque wave, obtained from Hottinger Baldwin Messtechnik Ltd., Darmstadt, Germany) served as the test bed for isometric torque measurements (FIG. 1). An 11-bit incremental encoder, synchronized to turn with the crankshaft, determined the actual position of the crank. Angular and force data were read in by the PC at a sample rate of 20 Hz.

The ankle joint was immobilized at 90° and leg movement was restricted to the sagittal plane using shank and foot orthoses. The crank angle was set and held automatically by an AC-servomotor (MR 7434 obtained from ESR Pollmeier Ltd., Ober-Ramstadt, Germany) position-controlled by a servo-controller (TrioDrive obtained from ESR Pollmeier Ltd., Oberramstadt, Germany). Volitionally or electrically evoked maximal isometric torques of the left leg were measured at a 100° crank angle (see FIG. 1, inset), with reference to the zero degree defined by the left, backwards-pointing crank arm (280° for the right leg, due to a shift of 180°.

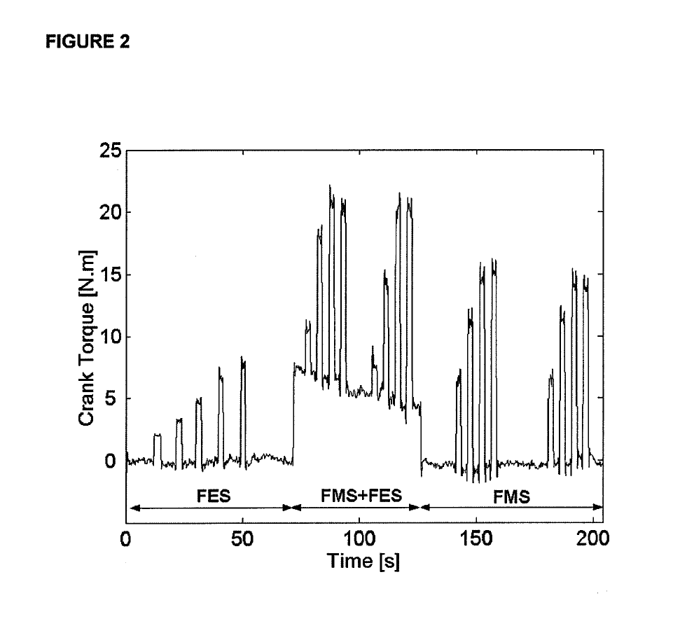

(A) Stroke group: The maximal torque generated by the quadriceps group was considered only on the affected side. After peak volitional torque on this side was recorded, the subject was instructed to relax for 20-30 s. Then beginning at the motor threshold, the muscle was stimulated by FES bursts (FIG. 2) with amplitudes increasing stepwise (5 mA) until the maximally tolerated intensity (indicated by the individual) was reached. Next, while the muscle had been electrically stimulated for 50 sec at the maximally tolerated FES intensity, FMS bursts that increased stepwise (15%) until the maximally tolerated FMS intensity was reached were applied to the muscle. Finally, the muscle was stimulated twice by the same sequence of FMS bursts as before. The peak torque and corresponding stimulation intensities were recorded in the sequence: FES, FMS+FES, and FMS (FIG. 2). The burst duration amounted to 1.5 s.

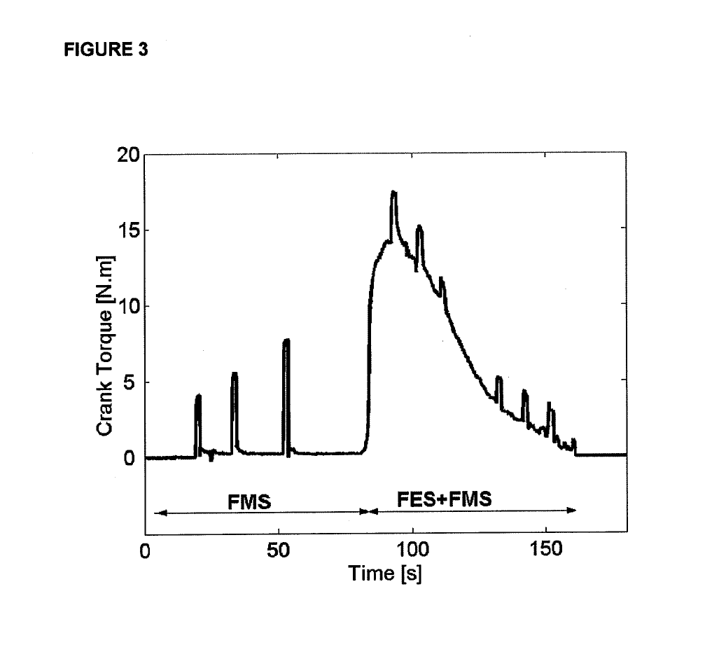

(B) SCI group: The maximal torque generated by the quadriceps group was recorded only at the left side. The muscle was successively stimulated by FMS pulses of amplitudes of 40%, 60%, 80%, and 100% and with a burst-duration of 1.5 s (FIG. 3).

Next, while the muscle had been electrically stimulated for 50 s at the maximal intensity, the same sequence of FMS bursts as used before was applied to the muscle. Peak torque and corresponding stimulation intensities were recorded in the sequence FMS, FES, and FES+FMS (FIG. 3).

1.6. Ergometric Measurements

Dynamic measurements were performed on the stationary tricycle test bed by controlling the resistance torque (motor-powered brake). The braking torque on the crank measured by the torque transducer ranged from 0.15 Nm to 7.30 Nm. It was set individually at the maximal magnitude at which the patient could cycle for about 3 minutes at a cadence of 35-55 rpm. FES and FMS were applied in a randomized order, each in a separate session.

(A) Stroke group: The subjects cycled for 3 minutes. This consisted solely of volitional cycling in alternation with stimulation-supported cycling, each time for periods of 30 seconds. Patients were instructed to try to achieve smooth pedaling. The maximally tolerable stimulation intensity, determined in isometric tests for each individual, was also used in the ergometric tests. Data for the last 15 seconds of the 30 s periods were collected for each individual and each condition (3 FES, 3 FMS, and 6 non-stimulated periods).

(B) SCI group: Data for two minutes of pedaling propelled by stimulation were recorded. The stimulation intensity was gradually increased over about 10-30 seconds to the maximum intensity (FES: 127 mA and FMS: 100%) while maintaining the cadence in the range of 35-55 rpm.

1.7. Data Processing

Crank angular position and torque were recorded: cadence and power were calculated (also cycling smoothness and symmetry in the stroke group). Cadence was computed from the change in crank position over time. This was digitally filtered with a second-order Butterworth filter with a cutoff frequency of 4 Hz. Power was defined as the product of cadence and torque.

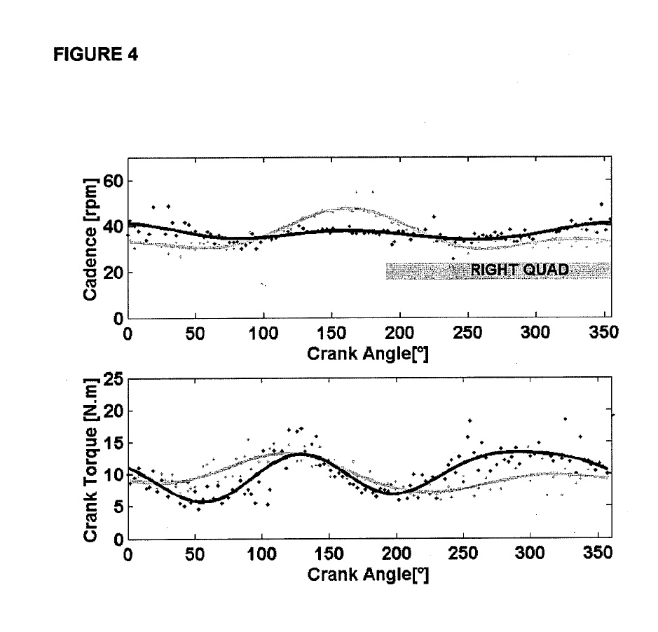

To measure the smoothness of reciprocal pedaling, a method proposed in the literature³¹ was used. The roughness index (RI), defined as the summation of the curvature for each instantaneous cranking speed, is given as:

$$RI = {\sum }_{1}^{360} \frac{d R}{d s}$$

where R is the instantaneous cranking speed after tenth-order polynomial curve fitting, and s is the crank position. In smooth pedaling, the RI will approach zero. The definition of the smoothness is illustrated by the upper graph of FIG. 4.

To measure cycling symmetry (Chen, H. Y. et al. (2005) J. Electromyogr. Kinesionol. 15, 587-595), the maximum of the circular auto-correlation coefficient of the crank torque profile (FIG. 4, lower graph) was calculated:

$$SI = {max}_{j} \vert {c}_{xx} ( j ) \vert$$

where is the angular lag between the two highest peaks of the crank torque profile taken over one pedaling cycle of 360°. The higher SI is as it approaches the maximum value of 1, the higher is the side symmetry in torque generation during cycling movement.

The polynomial regression and interpolation of the cadence and the torque to 1° crank angle of the pedaling cycle for the 30 s periods corresponding to each subject and condition were averaged together, yielding one cadence and torque profile (FIG. 4). By taking the mean value over the cycle, one observation of the power resulted for each subject and condition (FES, FMS, no stimulation). Roughness and symmetry were similarly processed for the stroke group.

1.8. Statistical Analysis

The isometric torque evoked and the power generated via electrical and magnetic stimulation were compared. Additionally, volitional and combined (i.e. electrically and magnetically) stimulated torques were considered for the stroke group as well as the smoothness and symmetry of pedaling in electrical and magnetic stimulation conditions. Statistical comparisons were made in the stroke and in the SCI group with the one-way repeated measures ANOVA test with the stimulation mode as factor (FES, FMS, and no stimulation) and with the paired t-test respective. Post hoc multiple comparisons in the stroke group were based on Tukey's honestly significant difference criterion. To determine the individual torque response variability to stimulation, linear correlation was used. Comparisons and correlation were considered significant at p<0.05. The analysis was performed with the Statistics Toolbox in Matlab V. 6.1.0 (obtained from Mathworks, Inc., Natick, Mass., USA).

### 2.1. Results of Isometric Measurements

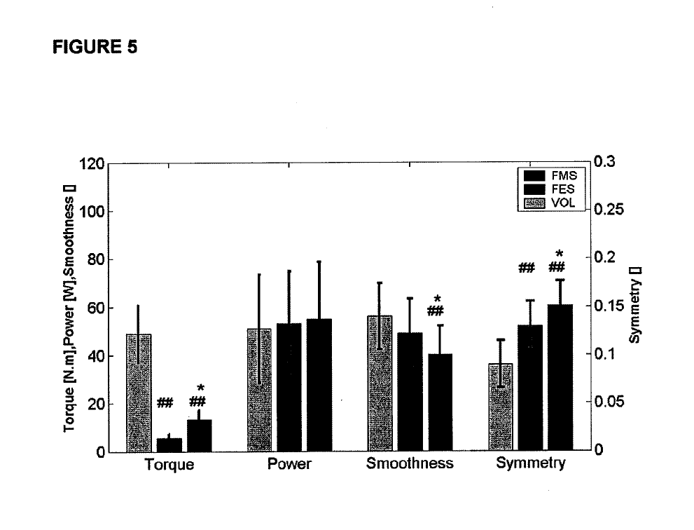

(A) Stroke group: Significantly more torque was produced volitionally (100%) than electrically (11%) or magnetically (27%) on the affected side of the study participants (p<0.001, see FIG. 5). Magnetically evoked torque evoked at 100% stimulation intensity was in all subjects higher than electrically evoked torque (at 62±33 mA intensity). FIG. 2 illustrates the complete stimulation protocol for a representative patient with stroke. FES produced a maximal isometric torque of about 7.5 Nm at a stimulation intensity of 75 mA. Using FMS, the torque achieved was about 15 Nm at 100% intensity. Combined application of FES+FMS evoked 20 Nm torque, i.e., the deviation from the sum of torques (15+7.5=22.5 Nm) amounts to only about 10%, showing that a summation effect of FES and FMS occurred. The torque produced by FES during combined stimulation showed some decay (about 30% in 50 seconds) caused by fatigue.

As a group, magnetic torque was significantly higher than electrical torque (13.4±3.8 Nm vs. 5.5±1.73 Nm, p<0.05; see FIG. 5). Investigation of individual response variability showed that a moderate correlation (r²=0.53, p<0.001) existed between electrically and magnetically evoked torques. The sum of electrical and magnetic evoked torques did not significantly differ from the combined (electrical+magnetic) torque (p=0.11).

(B) SCI group: Magnetically evoked torque (at 100% stimulation intensity) was in all subjects less than electrically evoked torque (at 127 mA intensity). While considerable fatigue occurred during continuous electrical stimulation at 127 mA (causing electrically evoked torque to vanish), typically positive torque pulses evoked by additionally pulsed magnetic stimulation at 100% occurred (FIG. 3). In a group comparison FMS produced significantly less isometric torque than FES (11.86±3.2 Nm vs. 16.6±3.5 Nm, p=0.003). Assessment of individual response variability showed that electrically and magnetically evoked torques correlated well (r²=0.71, p<0.001).

In some patients, maximal FMS bursts applied additionally to the FES produced negative break-ins instead of positive peaks.

In the group comparison, the sum of electrical and magnetic evoked torques differed significantly from the combined (electrical+magnetic) torque (p<10⁴).

2.2. Results of Ergometric Measurements

(A) Stroke group: Power generated during volitional, supported by FES and FMS amounted to 51.5±22.4 W, 53±21.7 W, and 55.2±23.4 W, respectively. Power did not show significant dependency on the cycling modality (p=0.79 in the ANOVA test).

In contrast, in a comparison of the symmetry and the smoothness of cycling with FMS vs volitional and also FES, the majority of subjects showed improvements both in symmetry (24 and 24 of 29, respectively) and in smoothness (27 and 21 of 29, respectively). In the case of the representative subject exemplified in FIG. 4, the right-sided hemiplegia caused asymmetrical torque production during purely volitional cycling (symmetry SI=0.07). Supportive FMS on the right side led to a larger symmetry SI=0.21. Smoothness improved as the roughness index RI decreased from 44 without FMS to 21 with FMS.

Likewise the smoothness of cycling in a group analysis (volitional 56±13.73, FES: 49±14.26, FMS: 40±11.97) was significantly improved (p<0.05) by FMS support. It did not significantly improve (p=0.65) with FES support compared to volitional cycling. Moreover, FMS-supported cycling was significantly smoother than FES-supported cycling (p<0.05).

As the smoothness improved, the symmetry increased significantly under FMS-supported (0.15±0.02) compared to volitional (0.09±0.02; p<0.001) or to FES-supported cycling (0.13±0.03: p<0.01).

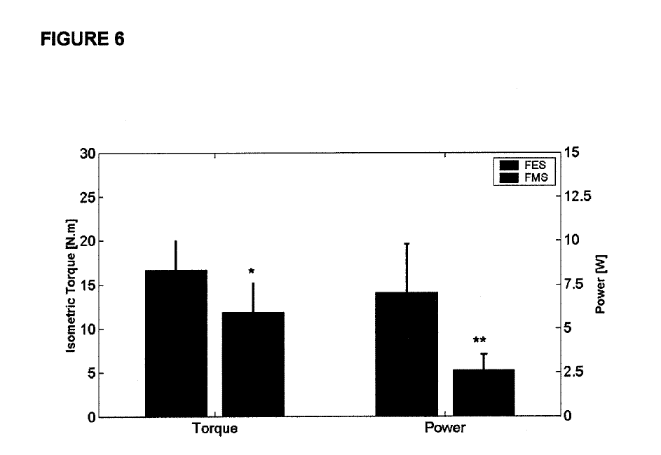

(B) SCI group: Although contiguous and smooth pedaling could be achieved in all subjects by magnetic stimulation (FIG. 1), less power was generated with FMS than with FES in all cases. Correspondingly, significantly less power was produced with FMS (2.61±0.88 W) than with FES stimulation (7±2.75 W, p<10⁻⁵; FIG. 6). This is in line with the above observation that less torque is generated by FMS than FES stimulation in fresh or moderately exhausted muscle.

3. Discussion

(A) Stroke group: The first important finding of this study, namely that under our conditions (devices, parameters, and stimulation sites) magnetic stimulation supports more effective cycling (in terms of more torque production and better dynamic parameters of cycling like smoothness and symmetry) of subjects with post-stroke hemiplegia than electrical stimulation does. This result is due to the partially or completely preserved sensibility in these individuals, which hinders the application of FES more than that of FMS.

Torques evoked by FES and FMS amounted in average to 5.5 Nm and 13.4 Nm, respectively. Therefore, the ratio of FES- and FMS-generated torque in the stroke group is comparable to a similar ratio found in 17 healthy persons by Han, T. R. et al. (2006), supra (mean isometric peak torques evoked by FES and FMS amounted to 4.4 Nm and 9.5 Nm, respectively).

While the FMS-produced torque represents a significant increase of the volitional torque (volitional+FMS compared to volitional; p<0.05), this is not true for the FES-produced torque. The magnetically and electrically produced torques correlated only moderately (r²=0.53), because individuals do not respond to both stimulation modes in the same manner. One can speculate that the torque-producing capability depends on the individual's muscle “intrinsic tissue properties” (Lieber, R. L. et al. (2004) Muscle Nerve 29, 615-627), and also on the pain tolerance of each individual.

In the ergometric experiment, the power did not increase significantly with any stimulation support. From the viewpoint of kinematic analysis, one would expect a smoother and more symmetrical pedaling with stimulation than without. However, this was achieved only with FMS, presumably because of the higher torques produced with magnetic stimulation.

The summation effect of combined stimulation (FES+FMS), which we observed in the quadriceps musculature of some subjects with stroke, could be interpreted as an additional torque produced by a new, fresh pool of muscle fibers being mobilized by additional magnetic stimulation (FIG. 2). A similar summation effect was described earlier (Garnham, C. W. et al. (1995) J. Med. Eng. Technol. 19, 57-61) in a healthy population who received stimulation of the ulnar nerve in a combined (FES+FMS) manner. Therefore, such combined stimulation could be a means to improve mechanical output in patients with preserved sensibility.

(B) SCI group: Although magnetic stimulation-propelled cycling was possible, it was less effective than electrical stimulation in terms of torque and power-generating capability.

In the combined stimulation (FES+FMS) produced torque, the contribution of FES was more important than the contribution of FMS in the fresh muscle of the SCI group (FIG. 3). This was the opposite of the situation found in the stroke group, where FMS made the main contribution of torque (FIG. 2). This is explicable in terms of the decay of the electrical field with distance; the decay is less pronounced if induced by magnetic stimulation than by surface electrodes. Thus muscle tissue can be stimulated at a greater depth with magnetic stimulation (Barker, A. T. et al. (1987) supra; Barker, A. T. (1991) supra). As a summation effect could be shown in the SCI group, we propose that only a few deeper, fresh muscle parts could be mobilized by adding magnetic stimulation to electrical stimulation (FIG. 3). Moreover, the occurrence of negative peaks suggests that parts of the antagonistic muscle were activated by FMS. The muscle tissue stratification and thickness which affect penetration depth of FES and FMS stimulation are presumably different in chronic SCI and chronic stroke patients.

Since both magnetically and electrically produced torques in persons with complete SCI correlated well (r²=0.71), contrary to the stroke group, they presumably respond to both stimulation modes in a more similar manner. Perhaps their torque-producing capability depends mainly on the individual's muscle ‘intrinsic tissue properties’ (Lieber, R. L. et al. (2004), supra).

3.1. Experimental Setup

Fixed stimulation sequences (FES and FMS) were designed for subjects with stroke and subjects with SCI, respectively, thus allowing isometric measurements with FES, FMS, and combined stimulation (FES+FMS) during the same session. Because the interventions were not assigned to each subject in a random order in the isometric measurement protocol, interference effects, mainly fatigue, had to be considered (Neter, J. and Wassermann, W. (1974) Applied Linear Statistical Models. Homewood, Ill.: Irvin, Inc.). The rationale of the stimulation protocols used is based on our observations made in preliminary experiments that FMS (FES) evoked higher torques than FES (FMS) in subjects with stroke (SCI). Therefore, the adopted stimulation protocols decreased the studied effect rather than increased it.

3.2. Stimulation Conditions

The results of the present study strongly depend on the electrical and magnetic stimulation conditions used. These factors can influence the torque produced and the pain perceived during FES.

While selecting stimulation parameters one has to consider that the present study focused on optimization of stimulation-induced movement (particularly cycling) rather than solely on maximization of isometric torque. Therefore, torque has to be maximized and fatigue and discomfort minimized at the same time. While the literature is equivocal on the choice of an optimal frequency of FES of the lower extremity regarding isometric force and sensed discomfort (e.g., 25 Hz (Han, T. R. et. Al. (2006), supra; Malezic, M. et al. (1994) Int. J. Rehabil. Res. 17, 169-179) and 30 Hz (Yan, T. et al. (2005), supra; Bogataj, U. et al. (1995) Phys. Ther. 75, 490-502), the usage of 20 Hz seems to be well founded³⁰.

Moreover, previous work performed in our laboratory on the FES cycling of persons with complete paraplegia showed that a stimulation of 20 Hz was superior to higher frequencies as regards average power produced during cycling. This is because higher frequencies cause more rapid fatigue (Naaman, S. C. et al. (2000) Neurorehabil. Neural Repair 14, 223-228). Furthermore, technical limitations of the magnetic stimulators require using stimulation at 20 Hz. Therefore, this frequency was adopted during both electrical and magnetic stimulation. Other parameters were set to provide maximal mechanical output according to our laboratory standard (FES pulse shape, maximal amplitude and width, coil placement) or fixed at today's technical standard (FMS induction shape, width, and maximal amplitude).

Electrode size and placement was similar to specifications in the literature (Han, T. R. et al. (2006), supra; Noel, G. and Belanger, A. Y. (1987), supra) but differed from others (Yan, T. et al. (2005), supra; Bogataj, U. et al. (1995), supra) This electrode localization is favored because it was in accordance with previous work (Szecsi, J. et al. (2007a) Med. Sci. Sports Exerc. 39, 764-780: Szecsi, J. et al. (2007b) Arch. Phys. Med. Rehabil. 88, 338-345; however, it might have influenced our results.

Further, current induced by magnetic stimulation is strongly dependent on both coil shape and orientation (Amassian, V. E. et al. (1989) Exp. Neural. 103, 282-289; Maccabee, P. J. et al. (1991) J. Clin. Neurophysiol. 8, 38-55). While two kinds of coils are in use (circular and figure-eight shaped coils), the latter cannot be used in deep muscle stimulation, because of the strong focalization of the induced eddy currents. To achieve mechanical output that overcomes realistic drive resistances, deep musculature like the quadriceps has to be stimulated relatively homogeneously, using a larger coil size, like the 90-mm diameter circular coil. Moreover, a combination of large circular coils (or perhaps elliptical or coils wrapped around the muscle) with decreased muscle selectivity and mechanically constrained trajectories of the legs (as in cycling) seems to be an adequate application of magnetic stimulation. Another benefit of magnetic stimulation is that no direct skin contact is needed, unlike electrical stimulation, and therefore the patient can remained clothed during treatment.

4. Conclusions

The results of this study suggest that magnetic stimulation is a potential alternative to surface electrical stimulation of the large thigh musculature with regard to stimulation-supported cycling of patients with partially or completely preserved sensibility (with, for example, post-stroke hemiplegia or multiple sclerosis). While the present study compared the biomechanical efficacy of magnetic and electrical stimulation, further studies have to be performed to determine whether long-term repetitive application of magnetic stimulation is therapeutically more advantageous than electrical stimulation.

The present invention illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by embodiments and optional features, modifications and variations of the inventions embodied therein may be resorted to by those skilled in the art, and that such modifications and variations are considered to be within the scope of this invention.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. Method for the physical therapy of a paretic patient, the method comprising:
(a) providing a training device, comprising
(i) at least one magnetic stimulator for applying functional magnetic stimulation to at least one paralyzed muscle or muscle group of said patient in order to induce a periodical movement;
(ii) at least one guiding element for restricting the degrees of freedom of the movement induced by applying functional magnetic stimulation; and
(iii) at least one resistance element for providing a resistance against the movement induced by applying functional magnetic stimulation;
(b) applying magnetic stimulation to at least on paralyzed muscle or muscle group of said patient in order to induce a movement;
(c) impeding the movement of said muscle or muscle group via the at least one resistance element; and
(d) determining the torque of the movement induced.

2. The method of claim 1, further comprising:
(e) adjusting the functional magnetic stimulation in such a manner that the torque of the movement induced is at least 1.25 Nm.

3. The method of claim 1, wherein the patient to be treated is a patient having an at least partially preserved sensibility in the paralyzed part of the body.

4. The method of claim 1, wherein the patient to be treated is a patient suffering from a condition selected from the group consisting of stroke, multiple sclerosis, cerebral paresis, traumatic brain injury, and complete (SCI-A) or incomplete (SCI-B, C, D) spinal cord injury.

5. The method of claim 1 wherein the periodical movement is a cyclic movement.

6. The method of claim 1, wherein the at least one guiding element is selected from the group consisting of a stationary cycle, an ergometer, a cross-trainer, a rowing machine, a robot, and an exoskeleton.

7. The method of claim 1, wherein the at least one magnetic stimulator comprises any one or more of the group consisting of:
(i) one or more coil(s) selected from the group consisting of a ring coil, an elliptic coil, a saddle coil, a sleeve coil, a manchette coil or a semicylindrical coil,
(ii) an acoustic attenuation or damping element; and
(iii) a cooling system, the cooling system being configured to allow the continuous operation of the at least one magnetic stimulator for at least 5 minutes at a frequency of at least 20 Hz.

8. The method of claim 7, wherein the one or more coil(s) of the at least one magnetic stimulator is/are arranged in a latero-ventral position relative to the at least one paralyzed muscle or muscle group to be stimulated.

9. The method of claim 7, wherein the one or more coil(s) of the at least one magnetic stimulator is/are configured to apply magnetic stimulation to a body surface area of at least 250 cm².

10. The method of claim 9, wherein the body surface covers the quadriceps muscle group of the patient to be treated.

11. The method of claim 1, wherein the functional magnetic stimulation is continuously applied to the at least one paralyzed muscle or muscle group of the patient for at least 5 minutes during a treatment regimen.

12. The method of claim 1, wherein the functional magnetic stimulation is applied in at least three bouts within one day to the at least one paralyzed muscle or muscle group of the patient during a treatment regimen, each of the at least three bouts comprising a continuous application for at least two minutes.

13. Training device for the physical therapy of a paretic patient, comprising:
(a) at least one magnetic stimulator for applying functional magnetic stimulation to at least one paralyzed muscle or muscle group of said patient in order to induce a periodical movement;
(b) at least one guiding element for restricting the degrees of freedom of the movement induced by applying functional magnetic stimulation; and
(c) at least one resistance element for providing a resistance against the movement induced by applying functional magnetic stimulation;
wherein the device is configured in such a manner that the torque of the movement induced is at least 1.25 Nm.

14. The device of claim 13, wherein the periodical movement is a cyclic movement.

15. The device of claim 13, wherein the at least one guiding means is selected from the group consisting of a stationary cycle, an ergometer, a cross-trainer, a rowing machine, a robot, and an exoskeleton.

16. The device of claim 13, wherein the at least one magnetic stimulator comprises any one or more of the group consisting of:
(i) one or more coil(s) selected from the group consisting of a ring coil, an elliptic coil, a saddle coil, a sleeve coil, a manchette coil or a semicylindrical coil;
(ii) an acoustic attenuation or damping element; and
(iii) a cooling system, the cooling system being configured to allow the continuous operation of the at least one magnetic stimulator for at least 5 minutes at a frequency of at least 20 Hz.

17. The device of claim 16, wherein the one or more coils are arranged at a distance of at last 5 mm from the body surface area where the stimulation has to be applied.

18. The device of claim 16, wherein the one or more coil(s) of the at least one magnetic stimulator is/are arranged in a latero-ventral position relative to the at least one paralyzed muscle or muscle group to be stimulated.

19. The device of claim 16, wherein the one or more coil(s) of the at least one magnetic stimulator is/are configured to apply magnetic stimulation to a body surface area of at least 250 cm².

20. The device of claim 19, being configured to apply magnetic stimulation to the body surface covering the quadriceps muscle group of the patient to be treated.
